(19) United States
(12) Patent Application Publication
(10) Pub. No.: US 2025/0186227 A1
(43) Pub. Date: Jun. 12, 2025

(54) KIT HAVING A DEVICE FOR CREATING A PROSTHESIS SHAFT FROM UV-CURING RESIN MATERIAL, AND METHOD

(71) Applicant: ROMEDIS GMBH, Neubeuern (DE)

(72) Inventor: Andreas RADSPIELER, Neubeuern (DE)

(21) Appl. No.: 18/844,939

(22) PCT Filed: Mar. 7, 2023
(86) PCT No.: PCT/EP2023/055790

§ 371 Date: Sep. 6, 2024

(30) Foreign Application Priority Data
Mar. 7, 2022 (DE) 10 2022 105 269.9
Jan. 20, 2023 (DE) 10 2023 101 468.4

(51) Int. Cl. A61F 2/50

(52) U.S. Cl. CPC A61F 2/5046; A61F 2002/5053

(57) ABSTRACT

A set for taking an impression of a limb stump or in generating a prosthesis shaft or a part thereof, comprises or consists of a medical apparatus, at least one energy source and a deformable material which may be cured by an energy source. The medical apparatus comprises at least one pressure vessel with a fluid chamber or a pressure chamber for receiving or storing a fluid, wherein the pressure vessel comprises a wall made of a first material and a fluid-tight membrane made of at least a second material, and an insertion opening for inserting the limb stump into the interior of the pressure vessel. The membrane is arranged to form or delimit the fluid chamber or the pressure chamber. The energy source is used to cure or partially harden the deformable material in order to achieve an impression of the limb stump or to produce a prosthesis shaft.

### CROSS-REFERENCE TO RELATED APPLICATIONS

The present application is a U.S. National Stage Application of International Application No. PCT/EP2023/055790, filed on Mar. 7, 2023, which claims the benefit of German Application No. 10 2022 105 269.9, filed Mar. 7, 2022, and German Application No. 10 2023 101 468.4, filed Jan. 20, 2023, each of which are incorporated herein by reference in their entireties.

### FIELD OF THE INVENTION

The present invention relates to: a set for use in taking an impression of a limb stump, in particular of a lower leg stump, or in generating a prosthesis shaft or a part thereof;

a method for fitting a material being deformable and curable by an energy source to a limb stump, or for creating a prosthesis shaft, or part thereof, for a limb stump of a patient; a prosthesis shaft, or a part thereof made in an apparatus of the aforementioned set or according the aforementioned method; a membrane for use in the aforementioned set or method; a material which is deformable and curable by an energy source; a kit comprising the aforementioned material and a bag in which the material may be, or is stored; and a liner for use in the aforementioned set or method.

### BACKGROUND OF THE INVENTION

Leg amputees may recover mobility using leg prostheses. Modern leg prostheses encompass various modules which include the prothesis shaft.

### SUMMARY OF THE INVENTION

The present invention relates to the technical field of creating a prosthesis shaft of a prosthesis or creating a prothesis, preferably for the lower extremities, i.e. for a leg prosthesis, in particular a lower leg prosthesis.

An object of the present invention may be to propose a set with a medical apparatus for use in taking an impression for a prosthetic shaft or in generating a prosthesis shaft, or at least for an inner or outer shaft thereof, in particular for the lower extremity.

The object according to the present invention may be achieved by a set with the features as disclosed herein. It may also be achieved by a method with the features as disclosed herein and by a prosthesis shaft with the features as disclosed herein and by a membrane with the features as disclosed herein. A material with the features as disclosed herein and a kit with the features as disclosed herein may also serve to achieve the object according to the present invention.

According to the present invention, a set is thus proposed for use in taking an impression of a limb stump, in particular a lower leg stump, or for use in generating a prosthesis shaft, or a part thereof. The set comprises a medical device (hereinafter in short: apparatus).

The apparatus maybe used as a basis e.g. for generating a lower leg prosthesis shaft (but also other prostheses such as e.g. arm prostheses, thigh prostheses), on the, only preferably standing, patient.

In this, the apparatus comprises a fluid container or a pressure vessel with exactly or at least one fluid chamber.

This may receive or store an optionally pressurized fluid. In this, the pressure lies above the atmospheric pressure. The fluid is a gas or a liquid, preferably air or water, since the latter two are respectively cheap and easily available. The pressure vessel comprises a wall, which is made of at least or exactly one first material, or comprises at least one first material.

The wall of the pressure vessel limits its interior. According to the present invention, the interior of the pressure vessel is understood to be the space or volume defined by the geometry of the pressure vessel or encompassed or circumscribed by an outer wall of the pressure vessel. If the pressure vessel is for example cylindrical, then the interior of the pressure vessel is the space delimited by the cylindrical shell surface and the two end sides or end planes. If the pressure vessel is in another example rectangular, then the space of the interior is defined by the result of the multiplication of the height, width and depth of the rectangle. In determining the interior, it is irrelevant whether or not the space corresponding to the interior is fluid-tight. The interior does not describe a fluid-tight closed space but a volume circumscribed by the wall. The space which does not belong to the interior of the pressure vessel is referred to herein as its exterior.

The pressure vessel comprises an insertion opening through which the limb stump, which may in this respect herein also be understood as the distal end of the stump instead of the whole stump, may be inserted into the interior of the pressure vessel. The insertion opening may be, for example, an open end side or end plane, a through-opening in the wall or an opening which breaks through or interrupts the wall. In the area of the insertion opening, the interior of the pressure vessel is thus not separated from the exterior by a section of the wall. The insertion opening may lie in an inlet opening or in an insertion plane, through which the limb stump is inserted into the interior of the pressure vessel.

Furthermore, the pressure vessel comprises at least one or exactly one, preferably fluid-tight, membrane. Alternatively, the pressure vessel does not comprise such membrane as described further below, but only correspondingly suitable and/or provided receiving devices (such as the connector described in the following as optional) for receiving the membrane on/at the pressure vessel.

The membrane is made of, or comprises, a second material. The first and the second material differ from each other.

Optionally, at least one section of the membrane, which lies in an upper and/or a proximal area and/or which faces the insertion opening of the pressure vessel, is connected to at least one connector, preferably releasably. By using this at least one optional connector, the membrane is fastened, preferably releasably, to the pressure vessel. The connector may be ribbon-like, rope-like, flexible and/or bendable.

The set according to the present invention optionally further comprises at least one energy source for curing or partially curing a deformable material in order to take an impression of the limb stump or to create a prosthesis shaft.

Furthermore, the set according to the present invention optionally comprises a material which is initially deformable, e.g. manually and without the use of tools, which material is nonetheless curable by an energy source. After curing, this material is preferably no longer deformable, in particular not chip-free, permanent or remaining and/or not deformable without the use of tools, flexible, fluid, elastic and/or bendable, etc. The material is preferably not plaster (e.g. in the form of a plaster bandage), does not comprise plaster, or preferably does not consist solely of plaster and/or supporting material (fabric, mesh, gauze, etc.) required or normally used to hold moist plaster.

It is optionally encompassed by the present invention that the set comprises the medical apparatus, the energy source and the material.

The medical apparatus may be provided for use in taking a negative impression of a limb stump, in particular a lower leg stump, wherein the apparatus may comprise a fluid container (may be a pressure container) for a liquid, such as water, wherein the following may apply: The fluid container comprises an upper, or first, end side when the apparatus is in use and a lower, or second, end side when the apparatus is in use. In the area of the second end side, the fluid container may be closed in a fluid-tight manner, for example by a base area. The apparatus comprises a film or membrane for receiving a section of a limb stump. The membrane is preferably connected to the container in a fluid-tight manner and/or closes the container, or parts thereof, in a, at least in sections, fluid-tight manner at least at the top or at least at the first end side. The fluid container may optionally comprise a pressure chamber. The fluid container optionally comprises at least one outlet for the fluid, which is optionally provided with a valve or a stopcock for opening and closing the outlet. The membrane is preferably bag-like (i.e. with a dead end like a bag, as opposed to a tube open at the top and bottom). Optionally, at least an upper and/or proximal section and/or a section located in the area facing the insertion opening of the pressure vessel is fastened, for example by at least one connector, to or with a section of a wall of the fluid container, for example a base area or side surface thereof, preferably releasably, directly or indirectly. The membrane is preferably made of or comprises a material which has no (or only slight) elasticity or preferably no (or only slight) stretchability at least in a first direction of the material, preferably in a longitudinal direction of the container or in an insertion direction of the limb stump. Instead of having the aforementioned membrane and/or the connector, the apparatus may be designed or configured to be connected to a membrane and/or to a connector. Suitable connecting devices for this purpose may be provided, for example, on a base area and/or side wall.

According to the present invention, there is proposed a method for adapting a deformable material curable by an energy source, which may be a radiation source, to a limb stump or for creating a prosthetic shaft (or a part thereof) for a limb stump of a patient.

The method encompasses the step of providing a set according to the present invention.

Furthermore, filling the fluid chamber or pressure chamber of the pressure vessel with a fluid, in particular a liquid, is encompassed by the method according to the present invention if the pressure vessel of the medical apparatus is not already filled with fluid, preferably a liquid. Alternatively or additionally, a change of a liquid level within the fluid chamber or pressure chamber such that the membrane is covered by the liquid at least in sections thereof around the entire circumference of these sections, or such that the membrane bulges beyond the insertion opening of the pressure vessel into the exterior of the pressure vessel is optionally encompassed. Further alternatively, providing a fluid chamber or pressure chamber filled in such a manner, or otherwise suitably filled, is provided.

According to the present invention, a prosthesis shaft, or a part thereof, such as an outer shaft or an inner shaft, is further proposed, which has been created in an apparatus of the set according to the present invention or according to a method according to the present invention or has been cured in whole or in part, or which comprises or consists of the material according to the present invention.

According to the present invention, there is further proposed a membrane for use in a set according to the present invention and/or in a method according to the present invention, wherein the membrane is transparent and/or comprises or is connected to a radiation source or light source. There is further proposed a use of such a membrane as disclosed herein for the membrane.

According to the present invention, a material is proposed which is deformable and curable by an energy source.

According to the present invention, there is further proposed a liner as disclosed herein, which is also an independent invention.

Furthermore, according to the present invention, a kit is proposed which consists of or comprises a material according to the present invention and a bag or other receptacle such as a case, a box, etc., or the like, in which the material according to the present invention is or may be stored.

Embodiments according to the present invention of each of the above-mentioned subject-matters may have one or more of the features mentioned below in any combination, unless a specific combination is obviously technically impossible for the skilled person. The subject-matters of the dependent claims each specify embodiments according to the present invention.

In all of the aforementioned and following statements, the use of the expression “may be” or “may have”, “can be” or “can have” and so on, is to be understood synonymously with “preferably is” or “preferably has”, and so on respectively, and is intended to illustrate embodiments according to the present invention.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”.

This understanding is also equally encompassed by the present invention as the interpretation that a numerical word, for example, “one” (or “a/an”) may alternatively mean “exactly one”, wherever this is evidently technically possible in the view of the person skilled in the art. Both of these understandings are encompassed by the present invention and apply herein to all used numerical words.

Whenever spatial references such as “top”, “bottom”, “upper” or “lower” are mentioned herein, the skilled person understands these in case of doubt as a spatial information with reference to the orientation in the figures attached hereto and/or the arrangement of the set according to the present invention with its apparatus(es) in its intended use.

Whenever an embodiment is mentioned herein, it represents an exemplary embodiment according to the present invention which is not to be understood as limiting.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

In certain embodiments, the pressure chamber is a locked and/or lockable space in which the fluid may be subject to pressure above the atmospheric pressure (in short: atmosphere) without being able to escape from this space.

In some embodiments, the membrane is arranged to form the pressure chamber or alternatively to delimit it, for example, by being part, in particular by being elastic or only in one direction (e.g. the circumferential direction) elastic part, of the wall or of the limitation of the pressure chamber.

Since it can receive and/or hold fluid under a pressure which is above atmospheric pressure, this fluid chamber is designated herein as pressure chamber.

The terms “fluid chamber” and “pressure chamber” are interchangeable in certain embodiments and/or in those embodiments in which the person skilled in the art does not object thereto. What is said herein about the “pressure chamber” may also be applicable to a “fluid chamber”.

In some embodiments, the pressure chamber then lies inside the pressure vessel when the pressure prevailing in the pressure chamber does not exceed a certain pressure. Deviating from this, in certain embodiments, the pressure chamber extends also to the exterior of the pressure vessel when the pressure prevailing in the pressure chamber pressurizes the membrane such that the latter projects outwards, for example through the insertion opening, i.e. into the exterior of the pressure vessel. The pressure chamber may thus have a variable volume, which depends on the pressure prevailing in the pressure chamber. The latter statement does not apply to the constant inside or inner volume of the pressure vessel.

In some embodiments, a fluid may be maintained in the pressure chamber under a pressure above the atmosphere, despite the insertion opening penetrating through the wall.

In certain embodiments, the pressure chamber serves for receiving or supporting the distal end of the patient's limb stump which is inserted into the interior of the pressure vessel. Due to the fluid contained in the pressure chamber, the membrane nestles laterally or circumferentially to the distal end of the limb stump or to the entire limb stump. In this way, it may be possible to pressurize the limb stump in the area of, if desired, the entire material layer surrounding it, plaster bandage, etc. throughout the membrane, with a-referring to an area unit-preferably unchanged or the same pressure. The latter may be an advantageous contribution of the present invention, since the even pressurization may lead to an even modeling of the deformable, preferably curable material or of the moist plaster bandage. The contacting of the membrane effected by the fluid stored in the pressure chamber may advantageously make manual modeling unnecessary or may significantly reduce the necessary effort therefor.

In some embodiments, the pressure chamber is a fluid-tight closed space which is entirely, or among others, formed or limited by the wall of the pressure vessel and the membrane. In some embodiments, the term “pressure chamber” may be replaced by the definition above.

In some embodiments, the pressure vessel is the vessel or space in which the pressure chamber is arranged.

In several embodiments, the pressure vessel is a water vessel.

In some embodiments, the term “pressure vessel” may be replaced by the term “fluid vessel” or “water vessel”.

In certain embodiments, the pressure vessel has a cylindrical form.

In some embodiments, the pressure chamber is designed and/or arranged such that the pressure prevailing therein depends, inter alia or exclusively, on the insertion depth of the limb stump into the interior of the pressure vessel, however during the intended use of the apparatus and with closed inlets and outlets, if present.

In certain embodiments, the membrane, for example a second section of the membrane, in particular a section with increased strength (for example by increased wall thickness and/or other material properties due to composite materials, coatings or the like) compared to the other section(s) of the membrane, is arranged in the area of the insertion opening and is optionally there directly or indirectly connected to the wall in a releasable or non-releasable manner, preferably fluid-tightly. It closes the insertion opening preferably similar to a cap, insofar the pressure present in the pressure chamber does not deviate significantly from the atmospheric pressure.

In several embodiments, the membrane is arranged to prevent a fluid exchange or material exchange in the interior of the pressure vessel in its axial direction.

In certain embodiments, the membrane is always arranged as a single layer in axial and/or radial direction, in other embodiments, it is not.

In several embodiments, the membrane is directly or indirectly connected to the pressure vessel on a first end side of the pressure vessel, but not also to a second end side opposite to the first end side, however, not without connectors, arranged between membrane and end side, preferably with at least 5 cm length.

In certain embodiments, the membrane is flat or balloon-like (i.e. open at one end), but not tube-like (i.e. open at both ends).

In some embodiments, the membrane is permanently connected to the apparatus. Permanently means in certain embodiments according to the present invention that the membrane cannot be released from the pressure vessel without the use of tools or only destructively; for example, it can be permanently and yet releasably connected to the wall by a clamping ring or by clamping rings (not to be confused with connectors in the sense of the present invention, which may additionally be provided) and by one or more screws. A releasability using a tool may advantageously be provided in order to enable replacing the membrane, for instance due to abrasion after a plurality of uses. In these embodiments, however, the membrane is not intended to be releasable from the pressure vessel by simple pulling over, pulling down or the like.

Simultaneously, the permanent fastening may advantageously ensure that the forces which are transmitted in the pressure vessel by the fluid to the membrane during use of the apparatus cannot release the membrane from the pressure vessel nor from its wall.

In several embodiments, the apparatus is designed to take a negative impression of the limb stump. The negative impression has a wall thickness of preferably 2 mm to 8 mm, particularly preferably 3 mm to 6 mm. It may consist of, or comprise, the curable material; the method according to the present invention encompasses this as well.

In several embodiments, the method encompasses manufacturing or generating a prosthesis shaft, a part thereof, or a complete, i.e. ready-to-use, prosthesis based on the cured material without having previously taken a positive impression of the limb.

In some embodiments, the method does not encompass taking a plaster impression of the limb stump.

In certain embodiments, the prosthesis shaft has a wall thickness of preferably 2 mm to 12 mm, preferably 3 mm to 8 mm, more preferably 4 mm to 6 mm.

In some embodiments, the at least one connector is fastened to a base area, to the wall and/or to the second end side of the pressure vessel, preferably by one of its ends or end sections, while the other end or end section is preferably connected to the membrane.

By using this fastening, the connector may fix the membrane inside the pressure vessel so that the membrane cannot bulge or protrude outwards through the insertion opening, in particular for the advantageously simple insertion of a limb stump into the interior of the pressure vessel.

The connector may be fastened to the base area, the wall and/or the second end side of the pressure vessel by mechanical fastening devices, for example by hooks, clamp connections, screws, rivets or the like.

In several embodiments, the at least one connector is at least 10 cm long, in others at least 20 cm long, and again in others at least 30 cm long.

In several embodiments, the at least one connector does not serve for connecting the membrane in an area of the upper edge of the wall of the pressure vessel and/or for providing a fluid-tight connection between the membrane and the pressure vessel.

In some embodiments, the at least one connector is connected to a reinforced section of the membrane. A reinforced section of the membrane may comprise, for example, an increased wall thickness, other materials and/or material coatings.

A reinforced section of the membrane may comprise for example a greater strength. Increased forces, in particular frictional forces, on the reinforced section, for example when a limb stump is inserted into, or withdrawn from, the interior of the pressure vessel of the apparatus, may be advantageously dissipated via the connector by the reinforced section of the membrane.

A further section of the membrane which is not, at least not completely, exposed or subject to these forces may have other material properties, for example, to enable a more comfortable and softer fit of a limb stump in the membrane in the pressure chamber during the taking of an impression. In particular, the distal areas of a limb stump are often particularly sensitive to pressure and should therefore be relieved of pressure as much as possible during the taking of an impression.

In some embodiments, the reinforced section of the membrane is connected to the at least one connector on the one hand and to the wall or bottom of the pressure vessel on the other hand.

In some embodiments, at least two connectors are fastened to the circumference of the membrane. Similarly, three, four, five, six or more connectors may be fastened to the circumference of the membrane. The connectors may be fastened to the circumference of the membrane in regular or irregular distances to each other.

In several embodiments, the pressure chamber of the pressure vessel comprises water or another liquid in its interior or they are filled with this. Due to its lack of compressibility, a liquid leads to more reproducible results when taking the impression. Thereby, water is the cheapest and most-easily available variant.

In certain embodiments, the wall of the pressure vessel is transparent in at least sections thereof. This may allow visual monitoring of the insertion depth and other aspects when taking the impression by the orthopedic technician in a simple manner. In addition, light, e.g. UV light, may enter.

In some embodiments, the pressure vessel comprises one or more markings in the area of at least one transparent section thereof. The insertion depth of the limb stump may be read at said markings.

In some embodiments, the pressure chamber is formed or limited by at least a portion of the wall and by the membrane. The membrane is connected to the section of the wall, preferably in the area of the second end side of the pressure vessel, in a material connection, force-fit connection and/or form-fit connection. There may be a connector, or a connecting device, provided for this purpose. By the connector, an undesirable bulging, wandering, floating or stretching of the membrane towards the top or into the exterior of the pressure vessel, in which the membrane-unlike in the interior of the pressure vessel-is not laterally supported by the wall, may in some embodiments according to the present invention be prevented or limited to an acceptable dimension. The connector thus holds the membrane, at least substantially, optionally inside the pressure vessel. This, or the connector, may counteract an undesirable floating of the limb stump. The floating may have an unfavorable influence on the pressure prevailing in the pressure chamber and the pressure acting on the limb stump by the membrane, in that the membrane no longer contacts the stump with uniform pressure in all sections in which it surrounds the stump. Hence, however, the optimal pressures do not lie at the stump when taking the impression, therefore, leading to the fact that the taken impression has not been taken under subsequent loads, which occur when walking with the prosthesis to be manufactured. Furthermore, reduced or prevented floating may contribute to protecting the membrane which is protected against damage in the interior of the pressure vessel through the wall of the latter. There is advantageously no need for any other limitation of the floating of the membrane, e.g. by ring which engages tightly to the thigh, when the connector is provided by the present invention. Since such a ring would have to be provided in a plurality of sizes in order to be able to take impressions, using the apparatus, for a plurality of differently thick limb stumps, the skilled person is offered a simplicity that is easy to understand. In addition to simplifying the use of the device, it also means a saving in material for rings, costs, and the like.

In certain embodiments, the membrane is connected to the pressure vessel in the area of the insertion opening or of the first end side of the pressure vessel, and additionally in a second area or section of the pressure vessel which is different therefrom. The second area may preferably lie in the interior of the pressure vessel and/or in the pressure chamber. The membrane may preferably be connected to the second area by the connector. The membrane may preferably be in contact only indirectly with the second area, namely optionally by the connector, i.e. preferably not having itself a direct contact with the second area, i.e. preferably not touching it.

In several embodiments, the second area is a peripheral section of the base area or of the second end surface or end side.

In several embodiments, the connector is not elastic nor stretchable.

In several embodiments, the connector is length-adjustable. Its length may be adjusted by a corresponding adjusting device, which is preferably adjustable from the outside of the pressure vessel. As a result, the distance between the distal end of the membrane and, for example, the base area, the lower end side or end surface of the pressure vessel is changed. This allows to adjustably arrange the membrane within the pressure vessel which in turn may allow an optimal adjustment of the apparatus of the set according to the present invention onto the specific limb stump regardless of its length.

In some embodiments the connector directly or indirectly connects the membrane to an end side of the pressure vessel which lies opposite to the end of the pressure vessel comprising the insertion opening, i.e. connecting it in particular to a lower end side, end surface or base area.

In some embodiments, the connector does not connect the membrane to a middle or central area of the end surface, end side or base area. This allows or supports a comparatively free arrangement of the limb stump inserted into the membrane within the pressure vessel, without the connector applying pressure over or through the membrane on distal sections of the limb stump.

In some embodiments, connecting is to be understood as form-fit connection and/or force-fit connection and/or material connection.

In some embodiments, the connector is arranged to keep a distance between the section of the membrane connected to the connector on the one hand and the section of the base area, lower end side or end surface, likewise connected to the connector, on the other hand within the specified limits. The distance may be, e.g., constant with a non-elastic connector.

In some embodiments, the membrane is directly connected to the wall. The connector may hereby be the result of a joining process, for instance an adhesive section, a rivet or the like

In some exemplary embodiments according to the present invention, the membrane touches, in the area of its connection, the base area, bottom side or lower end side; in other embodiments the membrane or material thereof does not touch the aforementioned.

In certain embodiments, the membrane is made of or comprises material which comprises in a first direction of the material another elasticity or stretchability than in a second direction which is different from the first direction, wherein the second direction may optionally be perpendicular to the first direction. In these or in other embodiments, the specific elasticity or stretchability in the first and/or second direction, respectively, is achieved through fibers, which the membrane comprises, which are e.g. embedded in silicone, a silicone matrix or another material, preferably a fluid-tight material. Such fibers may run in the first and/or second direction of the membrane, substantially run in the first and/or second direction and/or substantially act in the first and/or second direction. In this or in other embodiments according to the present invention, the different elasticities or stretchabilities may be additionally or alternatively achieved through other designs.

When the membrane is ready for use or is fastened as intended on the pressure vessel, the first direction may be an insertion direction of the limb stump or a longitudinal direction of the pressure vessel. The second direction may be in the angle, e.g. in the right angle, relative to the first direction.

Fibers which extend or run or act in the first direction are optionally not stretchable; or are stretchable only to a small extent. Optionally, they are less stretchable than optionally provided fibers which run, extend or act in the second direction. Fibers which run, act or extend in the second direction may, if present, be stretchable, they may be more stretchable or more elastic than the fibers of the first direction; their stretchability may optionally correspond to that of the fibers which extend in the first direction. The fibers running in the first direction are optional, so are the fibers running in the second direction. The stretchability or elasticity of the fibers may correspond or correlate to that of the membrane. The fibers may be made of or comprise nylon. hey may be made of tension-resistant material, in particular of material which is more tension-resistant than the material embedding it. Such a design, which e.g. prevents, or significantly limits, a longitudinal stretching (which may optionally be the first direction), allowing at the same time a stretching of the membrane in the circumferential or radial direction for adapting it to the limb stump, may also advantageously counteract an undesired floating of the limb stump. In addition, the relationship between the depth of the pressure chamber and the insertion depth within the membrane may advantageously remain the same. Also, the above-mentioned multiple piece sealing ring or thigh sealing ring may optionally be omitted. Further, it may be possible to generate, using only one membrane, prosthesis shafts according to the present invention at a whole row of limb stumps which are differently thick in the cross section. The membrane advantageously fits sufficiently tightly with thick as well as with thin stockings in order to prevent the membrane from throwing creases as described herein. The cross-section/circumferential stretchability which the membrane may comprise in several embodiments according to the present invention, even when the longitudinal stretchability is prevented or limited, advantageously contributes thereto.

In several embodiments, the membrane is partially, in at least one section thereof or completely or as a whole less than 2 mm thick, preferably less than 1.5 mm, particularly preferred less than 1 mm thick. In some embodiments, the membrane has this thickness over at least 90% of its length, preferably over at least 95% of its length (in the longitudinal direction during use). At this thickness, it distorts the difference between the actual geometry of the limb stump and the curable material in a negligible manner, at most.

In certain embodiments, the wall of the pressure vessel is made of plastic, for example Plexiglas or polycarbonate, or comprises one or more of these materials.

In certain embodiments, the connector, the membrane and/or the base area comprises at least one thread for directly or indirectly screwing the membrane or a component connected therewith to the pressure vessel.

In certain embodiments, the membrane is made of or comprises a material which comprises, in a first direction and/or in a second direction thereof, fibers embedded in a matrix or otherwise connected thereto.

In certain embodiments, the membrane, which optionally comprises a matrix, or its matrix is made of, or comprises, silicone.

In certain embodiments, some or all of the fibers have a wavy, curvy or zig-zag course.

In certain embodiments, the membrane is non-stretchable or non-elastic in a first and/or in a second direction thereof.

In certain embodiments, “non-stretchable” or “non-elastic” means that the modulus of elasticity of the respective component (connector, membrane, fibers, etc.) is at least above 700 N/mm², preferably above 1000 N/mm², especially preferably above 2000 N/mm².

In certain embodiments, “non-stretchable” or “non-elastic” means that a stretchability of the respective component (connector, membrane, fibers, etc.) is not more than 20%, preferably not more than 10%, preferably not more than 5%, particularly preferably not more than 2% of its length before the component tears or breaks.

In certain embodiments, the membrane, fibers thereof and/or the connector has a modulus of elasticity such as nylon.

In certain embodiments, in addition to the upper and/or proximal section of the membrane and/or in addition to the section of the membrane which lies in the area facing the insertion opening of the pressure vessel, also a lower and/or distal section of the membrane and/or a section which lies in the area of the membrane facing away from the insertion opening of the pressure vessel and/or facing a base area is directly or indirectly, preferably releasably, connected at, or connected to a section of, a wall of the fluid container, for example of a base area or side area thereof, by using at least one connector referred to herein as a distal connector.

What is stated herein regarding connectors, in particular regarding their material, length, elasticity, stretchability and other properties, in particular material properties, also applies to the connector herein referred to as a distal connector, unless this gives rise to any contradiction recognizable to the skilled person.

The distal connector may be designed and/or provided in any embodiments according to the present invention, as disclosed in WO 2016/135320 A1, the disclosure of which is hereby also made the subject-matter of the present application in its entirety by reference.

In some embodiments, the connector and/or distal connection are completely inside the pressure vessel without contact to the exterior of the pressure vessel.

In some embodiments, the pressure vessel and/or a line communicating herewith comprises a pressure gauge which determines or measures the pressure prevailing in the pressure chamber. The orthopedic technician may orientate themselves according to the measured pressure, for example, for the above-mentioned pressure increase.

In several embodiments, the membrane is deformable to the inside or to the outside, preferably elastically stretchable. In others, it is elastic in one direction; but it is not elastic or comparatively or substantially less elastic in a direction which is particularly perpendicular thereto.

The apparatus, or any component thereof, of the set according to the present invention, in particular the membrane and the membrane according to the present invention, may be provided with one or more features in any combination as disclosed in WO 2016/135320 A1, the disclosure of which is hereby made the subject-matter of the present application in its entirety by reference.

In some embodiments, the membrane is made of fluid-tight, in particular water-tight, material. It may be made of or comprise silicone. It may be made of or comprise fiber-reinforced silicone. A co-polymer or a rubber may be provided instead of silicone.

In several embodiments of the set according to the present invention, the at least one energy source is or comprises a radiation source, in particular a light source, preferably a UV radiation source.

In some embodiments, the at least one energy source comprises at least five, preferably at least six, eight or more, UV radiation sources.

In several embodiments, the UV radiation sources emit light with a wavelength of 405 nm, preferably radiation with a wavelength between 395 nm and 415 nm, particularly preferably radiation with a wavelength between 400 nm and 410 nm, or only such radiation.

In some embodiments, the at least one energy source for curing or partial curing is connected to a section of the pressure vessel.

In several embodiments, the at least one energy source for curing or partial curing is not connected to the medical apparatus, in particular not to a section of the pressure vessel.

In some embodiments, the at least one energy source is arranged outside the pressure chamber of the pressure vessel or of the medical apparatus.

In several embodiments, the pressure chamber of the medical apparatus includes water as a fluid. In particular, this may be distilled water, water obtained by reverse osmosis or water treated in a different way.

In some embodiments of the set according to the present invention, the medical apparatus or sections thereof comprises at least one section which is permeable to radiation, in particular light radiation or UV light radiation, in particular in the area of the wall of the pressure vessel, e.g. in the form of one or more windows.

In several embodiments, the medical apparatus or at least a section thereof, in particular the wall, is made of, or comprises, a translucent, UV-resistant acrylic glass, such as Plexiglas®.

In some embodiments, in particular of the set according to the present invention, the material is, or comprises, light-curing resin, preferably acrylic resin.

In several embodiments, in particular of the set, the material is, or comprises, a photopolymer.

In some embodiments, in particular of the set, the material comprises an odorant. This may serve to give the prosthesis shaft a pleasant odor in the state of use, for example, lemon freshness or the like.

In several embodiments, in particular of the set, the material comprises reactive additives. In certain embodiments, these may contribute to the fact that, after initiation by UV light, also areas cure that are not directly, or are less, irradiated with UV light.

In some embodiments, in particular of the set, the material is applied to and/or incorporated into a braiding, tube, sock or stocking.

The present invention further includes a braiding, a tube, a sock or a stocking or a mesh structure (these terms are used synonymously in several embodiments), in particular each as described herein, on or in which the material according to the present invention is applied and/or incorporated.

In some embodiments, in particular of the set, the braiding, tube, sock or stocking is elastic, at least transversely elastic or elastic in the circumferential direction, in order to be able to adapt to a plurality of limb stump circumferences of different patients in the best possible way, i.e. preferably as crease-free as possible. In this, it can also be elastic in the longitudinal direction, although this is not the case in certain embodiments. Additionally or alternatively to the optional elasticity in the circumferential direction, the braid, tube, sock or stocking may however be variable in its circumferential dimension by the use of fibers or strips, e.g. as described herein.

In several embodiments, in particular of the set, the braiding, tube, sock and/or stocking is multi-layered, i.e. comprises different layers.

In some embodiments, in particular of the set, the braiding, the tube, the sock and/or the stocking comprises, or consists of, several layers, preferably at least four, preferably at least five or six layers. In certain embodiments, these layers may also be referred to as a “sandwich structure”.

In some embodiments, the braiding, tube, sock, mesh structure and/or stocking has, e.g. on its inner side or facing it, a layer which is more finely meshed than some or all of the layers arranged radially further out. In this way, light from the radial direction may initially enter, comparatively, from the outside to the inside through the tightness of the fabric radially. The fineness of the mesh may in several embodiments thus increase towards the limb stump. In other embodiments, this may be exactly the opposite. The latter embodiments are particularly suitable for curing by radiating light from radially inwards to radially outwards.

In several embodiments, in particular of the set, several of the layers comprise or consist of glass fibers.

Since glass fibers have a high bending strength, tensile and compressive strength as well as a high resistance to chemical and biological influences at a relatively low density, they may advantageously provide the necessary strength of the finished shaft of the prosthesis. Furthermore, glass fibers have heat-insulating and water-repellent properties.

In several embodiments, the kit comprises a liner and/or a liner is used in the method according to the present invention, in particular as disclosed herein respectively.

In some embodiments, the liner may comprise or be connected to radiation sources, in particular light sources, or light guides, provided and/or suitable for curing the material according to the present invention. In these embodiments, it could also be referred to as a “curing liner”. In these embodiments, light thus radiates radially from the inside to the outside during curing within the membrane, i.e. from the liner towards the membrane, wherein the curable material is arranged between the liner and the membrane.

In several embodiments, the liners are present in several sizes in a set or kit or otherwise, for example exactly or at least in 3, 5, 10 or 20 different sizes. Preferably, this serves to have a suitable liner for each stump structure of the patient from the set or kit available for patient care.

In some embodiments, light, in particular UV light, may be guided by the glass fibers. In such embodiments, the glass fiber, or the glass fibers, may themselves be the energy source. Alternatively or additionally, the one or more glass fibers may serve as an energy line, e.g. a light guide. They may be connected to the energy source, e.g. the light source. They may emit the light themselves. Connectors, connections or the like which are necessary or suitable for this purpose may be provided and/or may also be encompassed by the present invention.

In several embodiments, a layer of the braiding, tube, sock and/or stocking or of the optional liner, in particular an outer layer preferably facing away from the patient's limb stump during intended use, may consist of, or comprise, energy-conducting, e.g. light-conducting glass fibers. By the light which is introduced into or conducted by or along this layer, it may be possible, for example by light emission, to prompt the material, in particular the resin, preferably acrylic resin, or the photopolymer, to cure. External light sources, or light sources shining into the medical apparatus or its pressure vessel from outside, may thus be omitted.

In order to be able to emit light better, any sheath of the glass fibers which may be present can optionally comprise, at least in section, light-transmitting apertures, windows and/or the like.

In several embodiments, by using the light conducted through or along the layers within the braiding, the liner, the tube, the sock and/or the stocking, which layers comprise glass fibers or emitted by them, the material, in particular the resin, preferably acrylic resin, or the photopolymer, may be prompted to cure.

The “sandwich structure” may advantageously favor a homogeneous structure of the prosthesis shaft and thus ensure increased wearing comfort for the wearer of the prosthesis shaft.

Providing several layers, which lie on top of each other in a radial direction, contributes to the fact that crossing points, e.g. of the mesh, of different layers do not tend to lie on top of each other or, if they do, that the resulting accumulation of material in the radial direction does not give rise to avoidable pressure points which impair the future wearing comfort for the patient or the process of generating.

In some embodiments, in particular of the set, several of the layers comprise or consist of a mesh section or braided section.

In several embodiments, in particular of the set, one of the layers, in particular the innermost layer, is made of, or comprises, an elastic fabric. Preferably, this is a white or light-colored fabric in order to increase the light yield. Preferably, in these embodiments, a layer facing the patient's limb stump is made of a very fine-meshed or fine-pored material in order to advantageously achieve the smoothest possible inner surface of the shaft. This may contribute to the later requirement for a step-free inner side, which is required for the application of a vacuum.

In certain embodiments, the liner may be wet, for example, soaked with water.

In several embodiments of the set, the innermost layer is designed to be light-reflecting or reflective on its outer side, for example, by a mirror film.

A mirror film may in particular increase the luminosity of the UV lamps and improve the penetration of the, optionally several, deformable layers which are curable by an energy source. This may also contribute to shortening the curing time.

In some embodiments of the set according to the present invention, this set comprises a liner, e.g. as disclosed herein, which is preferably designed to be light-reflective or reflective on its outside, e.g., by a mirror film. The patient may wear the liner on the skin. The liner may be arranged between the limb stump and the curable material.

In certain embodiments of the set, the outermost layer or a plurality of outer layers are of a coarse mesh structure, for example a Chinese finger trap structure, which is particularly suitable and intended to be translucent.

In some embodiments, the material is connected to a connection, in particular a screw connection, for distal prosthesis parts, in particular before it hardens, or the material surrounds said connection in sections or it is embedded in the material.

In some embodiments of the set according to the present invention, the membrane of the medical apparatus is at least in sections permeable to rays from the energy source, in particular to light, preferably UV light, for example translucent or transparent.

In certain embodiments of the set according to the present invention, this set comprises a control device which is configured to limit the energy source such that a temperature of 40° C. is not exceeded on the inside of the braiding, hose, sock or stocking. Corresponding measurements may be carried out in advance and the process may be controlled accordingly. If a temperature sensor is provided, a corresponding control system may be provided. This advantageously ensures that the patient does not get too hot when fitting the prosthesis shaft and, therefore, feels more comfortable. In addition, injuries to the skin caused by heat may be prevented in this way.

Additionally or alternatively, a temperature sensor may be used as part of a heat monitoring device.

In several embodiments of the method according to the present invention, the method encompasses the further step of inserting the limb stump into the sack opening or bulging formed by the membrane inside the pressure vessel such that the limb is surrounded by the membrane at least in sections thereof around its entire circumference, and such that the deformable material which is curable by an energy source is present between the limb stump and the membrane at least in section.

In some embodiments of the method, the material may be in contact with the membrane and/or the skin of the limb stump. Alternatively, the material may be separated from one and/or the other of these two by a separating layer, e.g. made of film. Likewise, the above-mentioned liner may be provided as a separating layer.

The liner disclosed herein may perform a cooling function. For this purpose, it may comprise cooling elements, connections for a cooling medium, such as a cooling liquid, there may be a cooling medium flowing through it, etc.

In several embodiments of the method according to the present invention, the limb stump may be surrounded or wrapped by the material already before it is introduced into the pressure vessel. Alternatively, the limb stump may first come into direct or indirect contact with the material in the pressure vessel.

In several embodiments of the method according to the present invention, the method encompasses as a further step a removal of the limb stump with the material surrounding it and a subsequent curing or further curing of the material using the energy source outside the pressure vessel.

In some embodiments, the method according to the present invention encompasses the further step of removing the limb stump with the material surrounding it after the curing of the material, using the energy source inside the pressure vessel.

In certain embodiments, the resin used only reacts when irradiated with UV light and only then cures.

In some embodiments, the set according to the present invention additionally comprises fabric stockings soaked with resin, in particular in various sizes, prefabricated and/or prepacked under vacuum, in light-impermeable bags.

Advantageously, when curing the material inside the apparatus, the resin irradiated, e.g., by the UV light source may cure without developing a strong odor and/or heat.

In some embodiments, the resin used is provided in different colors.

In several embodiments, the prosthesis shaft cured e.g. by UV light, may be used immediately as such, in particular without further manual processing, optionally at least as far as the prosthesis shaft as such is concerned. This may not effect the attachment of components that are required to obtain the ready-to-use prosthesis from the prosthesis shaft. Such components may relate to screws, valves, etc.

In some embodiments, it is provided to further process the cured prosthesis shaft, e.g. by UV light, in particular by thermal deformation.

In some embodiments, it is provided to equip the material to be cured, e.g. by UV light, with a direct distal screw connection to other prosthesis components, in others it is not.

In some embodiments, the membrane comprises a radiation-conducting, energy-conducting and/or light-conducting layer or a radiation-conducting, energy-conducting and/or light-conducting section.

In several embodiments, the membrane comprises radiation-emitting, energy-emitting and/or light-emitting layers or a radiation-emitting, energy-emitting and/or light-emitting section, or a layer or section that may be triggered to do so. Thus, it may be provided that the membrane, incorporated in it or in the material forming it (as e.g. disclosed herein), for instance between two layers thereof, comprises at least one light source or UV light source or other radiation device which is actuated as required and thus triggered to emit light, radiation, etc. There is encompassed e.g. the provision of UV emitters which are available within the membrane as light sources and which may be brought to emit light when required, but which otherwise preferably do not emit light.

In several embodiments, the apparatus comprises a voltage source, or a connection thereto, for supplying electrical voltage to the energy source or energy radiation source. In some embodiments, the voltage source is limited to a maximum output voltage of 12 V. The voltage source may be used as a voltage source for, e.g., the radiation unit or the light source.

In some embodiments, the apparatus comprises a mounting for at least temporarily fastening the radiation source to sections of the apparatus, e.g., to a wall thereof.

In some embodiments, the radiation source is an induction current source.

In several embodiments, the light sources or radiation sources comprise, or are in contact with, thermal accumulators, for example made of aluminum. They can counteract overheating of the radiation sources and thus contribute to increasing their service life.

In some embodiments, the set according to the present invention comprises, in addition to or as an alternative to the energy source and/or to the curable material, or the kit comprises, one or more form bodies or mechanical form bodies, for examples blocks or staples, e.g. supracondylar staples. These form bodies may be made, e.g., of or with carbon, plastic, fiber-reinforced plastic, in particular carbon fiber-reinforced plastic, or silicone. They may be provided and/or suitable for being applied directly to the patient's skin or placed and/or fixed there, for example using film. Thereby, they may reduce, e.g., the medial-lateral distance by applying pressure. The form bodies serve to shape the prosthesis shaft such that the wearing comfort of the finished prosthesis is increased for the patient, for example by relieving the patient's flexor tendons.

The form bodies may be designed so that to be changed in form or adapted to the patient, e.g., by heat. Their heat adaptation may be part of the method according to the present invention.

In some embodiments, the set comprises a calculation device. The latter may be part of the medical apparatus or connected to it physically or in signal communication. However, it may also be provided independently of the medical apparatus.

The calculation device, which may be a control device, may comprise an input device (for example, a keyboard, a scanner, a mouse, an interface, a stick, a memory unit, an interface (Bluetooth or the like), etc.) and an output device (for example, a monitor, a display, a printer, a memory unit, an interface (Bluetooth or the like), etc.) and/or a data memory.

The calculation device is optionally configured to monitor and/or limit-for example on the basis of data stored in the data memory or on the basis of input data which can be entered via the input device-technical parameters, for example a (minimum) duty cycle of the radiation sources or light sources, a total operating time thereof (for example, distributed over a predetermined time interval), a maximum duty cycle, a power of the radiation source(s), a predetermined temperature at the light sources and/or at the patient, a number of operating cycles or the like.

All the features and properties stated for the material of the set also apply without restriction to the material according to the present invention, and vice versa.

In some embodiments of the kit according to the present invention, the bag is a light-impermeable and air-impermeable bag.

In several embodiments, the kit comprises several such braiding, tubes, socks or stockings as described herein. These may be present in one or more bags.

In some embodiments of the kit, at least two, or more, of the braidings, tubes, socks, or stockings differ from each other in at least one corresponding circumference, e.g., at the same height, at an identical distance from a distal end, etc.

In several embodiments of the kit, at least two, or more, of the braidings, tubes, socks, or stockings have identical length (in distal-proximal orientation).

Several or all embodiments according to the present invention may have one or more of the advantages mentioned above or in the following.

By using the present invention, it is advantageously possible for the first time with a standing patient who subjects the stump to load to reliably and, above all, reproducibly not only take an impression of the patient's limb stump but also simultaneously generate a prosthesis shaft.

Thus, the present invention allows to take an impression as a basis for a shaft that is conveniently fitting the patient, especially when walking and standing. In this, less craftmanship skill is needed than hitherto required. Steps which were still necessary after obtaining a conventional plaster impression in order to achieve a prosthesis shaft may be omitted.

Thus, the present invention may render possible creating shafts for prostheses of the upper and especially lower extremities of humans in an objective manner. The present invention thus makes it possible to create a well-fitted prosthesis shaft, wherein it is possible to avoid the purely subjective and manually performed cost-intensive activities required by prior art methods.

Compared to the prior art, the present invention makes it possible to create shafts for prostheses in an objective manner. This ensures a better care or caring through improved fitting (form) and may reduce creation costs in that there is no, or only little, need for costly manual post-processing and adaptation. In addition, patient care may be accelerated, because also time-consuming adaptation steps may, at least in number, be greatly reduced or even completely eliminated.

A further advantage of the present invention may be that when using the curing material, e.g., the resin-soaked stocking, the cured shaft may be used immediately as a prosthesis shaft. Taking impressions and forms which is time-consuming and requires a great deal of manual skill, may be advantageously omitted. This helps to save time and costs for material and labor.

A further advantage of the present invention may be that the airtightness of the prosthesis shaft may be ensured by a suitable choice of the amount or ratio of laminate or braiding and resin. This may ensure a good fit of the prosthesis to the patient's limb stump without the need for further apparatus. This may help save time and costs and increase the wearing comfort of the prosthesis for the patient.

The deformable material that may be cured by an energy source, e.g., the resin with which the stocking is soaked, preferably does not reach more than 40° C. during curing, which advantageously ensures that it will not become too hot for the patient and that the fitting of the prosthesis shaft is perceived as comfortable by the patient.

Full curing of a prosthesis shaft may be achieved quickly, for example at 405 nm wavelength of UV light, in particular within 5 minutes or less, and without complications, which may also advantageously contribute to the comfort of the patient when fitting the prosthesis shaft.

A further advantage may be that, compared to other prior art creation methods, the production of a prosthesis shaft using the present invention may significantly avoid waste and save electricity and working time. For example, the inventor achieved savings of 85% waste, 92.5% electricity and 80% working time in generating a prototype compared to the conventional procedure.

A further advantage may be that a prosthesis shaft created by the present invention remains thermoplastically deformable even after curing, for example to allow an orthopedic technician to make minor adaptations to the patient's limb stump after the production.

### BRIEF DESCRIPTION OF THE DRAWINGS

The present invention is exemplarily explained below with reference to the accompanying drawings, in which identical reference numerals denote identical or similar components. In the figures, some of which are highly simplified, the following applies:

FIG. 1 shows a longitudinally cut medical apparatus of the set according to the present invention in a first embodiment from the side;

FIG. 2a, b show the apparatus of FIG. 1 in various embodiments in a further sectional plane;

FIG. 3 shows a longitudinally cut apparatus according to the present invention in a further embodiment from the side;

FIG. 4 shows a longitudinally cut medical apparatus of the set according to the present invention in a further embodiment from the side;

FIG. 5a shows a schematic representation of a braiding into or onto which a material is incorporated or applied, in an embodiment;

FIG. 5b shows a schematic representation of the braiding of FIG. 5a from the side;

FIG. 6a, 6b show a schematic representation of the possible principle of the braiding in an again further embodiment;

FIG. 7a shows a braiding in a further embodiment;

FIG. 7b, 7c show braidings with one or more mesh layers or braided layers, similar to the representation of FIG. 7a;

FIG. 8a shows a schematically simplified form body in a first embodiment;

FIG. 8b shows a schematically simplified cross-section of the limb stump with the form body of FIG. 8a from the top;

FIG. 8c shows schematically simplified a form body in a further embodiment;

FIG. 8d shows schematically simplified the form body of FIG. 8c in a top view; and

FIG. 9 shows schematically simplified an exemplary course of a method according to the present invention for adapting a deformable material, being curable by an energy source, to a limb; or for creating a prosthesis shaft or a part thereof for a limb stump of a patient, in one embodiment.

### DETAILED DESCRIPTION OF THE INVENTION

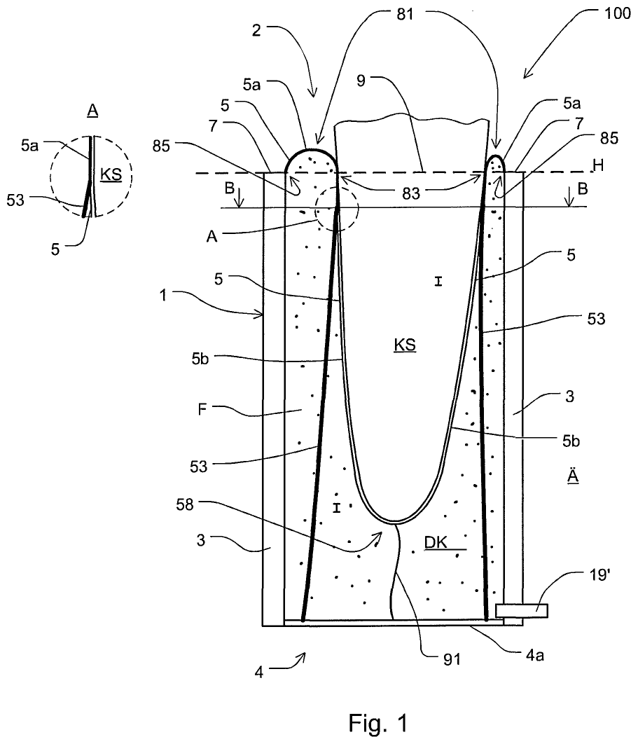

FIG. 1 shows an apparatus 100 of a set in a first embodiment according to the present invention, said apparatus 100 being cut longitudinally or in the longitudinal direction (i.e.

from top to bottom with regard to FIG. 1). “Proximal” lies correspondingly at the top, higher or in the direction towards it.

The first embodiment shows a pressure vessel 1 with a pressure chamber DK, a wall 3 and a membrane 5. The membrane 5 has a first or proximal membrane section 5a, which is connected to an upper edge 7 of the wall 3. In this embodiment example, the first membrane section 5a is optionally reinforced, which is shown by a thicker wall thickness (shown in FIG. 1 by a thicker line thickness). A reinforced design may alternatively or additionally be implemented by a different choice of material, a fiber-reinforced composite material, a material coating or in another way.

Furthermore, the membrane 5 comprises a second or distal membrane section 5b, which is arranged in the central section of the membrane 5 and is connected to, or integral with, the first membrane section 5a. Unlike the first membrane section 5a, the second membrane section 5b is optionally not reinforced, which is shown by a thinner wall thickness (outlined by a thinner line thickness in FIG. 1).

The two different membrane sections 5a, 5b serve for different load situations of the membrane 5. When inserting and removing a limb stump KS into the membrane 5, and thus into the pressure vessel 1, the first membrane section 5a in particular is loaded by frictional forces between the limb stump KS and the membrane 5. Due to this increased load, the first membrane section 5a is optionally made reinforced. The second membrane section 5b has in particular a sealing function and holding function for the limb stump KS in the pressure vessel DK with a lower load compared to the first membrane section 5a.

Two optional connectors 53, the function of which is described in more detail below, are optionally arranged in this exemplary embodiment at the connection site or interface between the first 5a and second 5b membrane section or in the transition area between the two. In particular, the connectors 53 are connected to the first membrane section 5a (optionally alternatively at a position other than that shown in FIG. 1).

The connection between connectors 53 and membrane section 5a or 5b is designated by the reference numeral A. In the example of FIG. 1, the connection A lies optionally below the height H and/or the upper edge of the wall 3 by the distance B.

The distance B may optionally lie between 0 cm and 40 cm, preferably between 5 and 30 cm, particularly preferably between 8 cm and 10 cm.

The area of connection A is shown enlarged on the left in FIG. 1.

The membrane 5 is fastened preferably releasably to the base area 4a, alternatively to an area of the wall 3 (for instance in its upper, middle or lower area), using the at least two connectors 53, which extend from the membrane 5 at a proximal section thereof.

The proximal area of the membrane 5 is to be exemplarily understood herein as the upper third of the membrane 5 or as an area in the upper third of the membrane 5.

The proximal area of the membrane 5 is recognizable facing the insertion opening 9 or is present in the vicinity thereof.

The area with which the membrane 5 is connected to the connector 53 is recognizably not located in a central, distal section 58 of the membrane. This advantageously prevents radial pressure onto the membrane 5 in a distal section due to the connector preventing the floating of the limb stump KS. This pressure could effect a radial compression of the limb stump KS stuck in the membrane 5 which may lead to an undesirable geometry of the generated plaster impression above all in the distal stump section. The provision of the connector 53 in a proximal section may help to avoid such deformations of the distal part of the limb stump This may advantageously result in a correct geometry of the plaster impression. Although the connector 53 effects a radial pressure onto the limp stump KS in a proximal section due to its proximal connection to the membrane, however, there (i.e. proximal or with regard to FIG. 1 “at the top”) the limb stump KS comprises a stronger anatomy and is less susceptible to radial compression which is avoidable by the present invention in some embodiments.

The pressure vessel 1 which is purely optionally cylindrical as shown in FIG. 1, comprises optionally a first end side 2 (at the top in FIG. 1) and a second end side 4 (at the bottom in FIG. 1). The second end side 4 in the exemplary embodiment of FIG. 1 is fluid-tight sealed with a bottom plate or base area 4a against an exterior Ä. The base area 4a may be made of the same material as the wall 3. It may be manufactured in one piece therewith.

The membrane 5 separates, in a fluid-tight manner, the fluid chamber or pressure chamber DK of the pressure vessel 1 from an exterior of the fluid chamber or pressure chamber DK, or exemplarily from an exterior Ä, i.e. an environment of the pressure vessel 1, or, as shown in FIG. 1, against a limb stump KS inserted into, or surrounded by, the membrane 5.

The membrane 5 may be fluid-tight connected to the pressure vessel 1 at an upper, often circular, rectangular, square or differently shaped circumferential edge 7 of the wall 3, or at another site.

The upper edge 7 is exemplarily present in a plane in which there is an insertion opening 9 of the pressure vessel 1 or it delimits said insertion opening 9 at its circumference. The insertion opening 9 is optionally present in the plane H which is denoted by a dashed line.

The insertion opening 9 serves inserting the limb stump KS which is surrounded with the curable material into an interior I of the pressure vessel 1.

The interior I is the volume of the pressure vessel 1 delimited by the wall 3. It extends from the second end side 4, which is fluid-tightly sealed with the base area 4a, to the insertion opening denoted with 9 and outlined by a dashed line.

The pressure chamber DK is filled with a fluid, here exemplarily with a liquid F outlined by points.

In FIG. 1, the apparatus 100 is illustrated in a state in which the extremely schematically outlined limb stump KS of the standing patient is inserted into the interior I such that it is surrounded by the membrane 5 at least in its distal section. The membrane 5 contacts the curable material (covering or on the limb stump KS) like a second skin, wherein there may be further layers, like liners or the like, provided between the curable material and the membrane 5.

The limb stump KS is in the example of FIG. 1 loaded with the full body weight of the standing patient. The amount of the liquid F is measured with regard to the known volume of the interior I or the pressure vessel 1 such that the limb stump KS may enter through the insertion opening 9 into the pressure vessel 1 at least so deep or far that the entire area, on which the curable material is present, contacts the membrane 5; at least as much as it is relevant for the creation of the prosthesis shaft. Simultaneously, the amount of liquid F is measured such that the distal end of the limb stump KS (at the bottom in FIG. 1) does not touch the bottom of the pressure vessel 1 or does not support itself on the bottom. In this way it is ensured that the patient rests with the inserted extremity on the pressure of the fluid and that the curable material is subject to the same pressure at every point by the membrane 5.

As is shown in FIG. 1, the membrane 5, when no limb stump KS is inserted into the pressure vessel 1, lifts up or floats due to the pressure of the fluid, here of the liquid, and a liquid level appears, not shown in FIG. 1. Hence, the shape of the membrane 5, which is shown in FIG. 1, represents the form which the membrane 5 adopts under loading when it fits closely to the inserted limb stump KS and is pushed under elastic stretching by the latter—in the example of FIG. 1—into the depth of the interior I towards the base area 4a.

It is further to be seen in FIG. 1 that due to the fact that both the wall 3 and the membrane 5 prevent a fluid exchange between the pressure chamber DK and the exterior Ä or a fluid leakage from the pressure chamber DK, they, hence, allow that the desired pressure builds up within the pressure chamber DK of the pressure vessel 1. However, they do not permit it to escape from the latter or to dissipate.

As is seen in FIG. 1, the pressure chamber DK is thus optionally formed by the membrane 5 and at least by parts of the wall 3 which, here, includes also the base area 4a of the end side 4.

Furthermore, FIG. 1 shows an optional outlet 19′ in the side wall or wall 3 of the pressure vessel 1. If provided, this outlet may also be positioned elsewhere at the pressure vessel 1, e.g. in its base area.

The outlet 19′ connects the interior I in the area of the pressure chamber DK to the exterior Ä of the pressure vessel 1. It allows the filling level and/or pressure within the pressure chamber DK to be specifically modified, for example by discharging or filling in fluid from the pressure chamber DK via the outlet 19′. For this purpose, the outlet 19′ is provided with a valve or stopcock not presented in detail which may be fluid-tight closed. Regardless of its designation as an outlet, the latter may also be used for introducing fluid and thus for filling the pressure chamber DK.

The membrane 5 is in the embodiment of FIG. 1 optionally connected purely exemplarily to the base area 4a by the connectors 53 or the connector 53. The connector 53 is optionally not (or only slightly) elastic or stretchable. The membrane 5 is optionally also not (or only slightly) elastic or stretchable in the longitudinal direction.

The connector 53 optionally allows the membrane 5 to float proximally (upward in FIG. 1) to a small and/or predetermined extent, or prevent same completely. Therefore, a more or less annular, however closed in its circumference, floating 81 arises at the second end side 2. The floating 81 is to be seen, due to the sectional illustration in FIG. 1, as protrusions left and right of the limb stump KS. The connector 53 may be provided in order to prevent such a floating 81.

The membrane 5 contacts the limb stump KS (or a layer pulled over the curable material, a liner or the like) closely at a contact point surface 83, which is more or less annular, however closed in its circumference. The contact point surface 83 is the circumferentially closed line or surface which comprises the points at which the limb stump KS has a final or closing proximal contact with the membrane 5. The contact point surface may also be referred to as contact area or contact line.

Simultaneously, the membrane 5, optionally at its radial circumference in the area of the second end side 2 of the wall 3, contacts, or is fixed with, a transition section 85 which is likewise closed in its circumference. The transition section 85 may be understood to be an area in which the limitation of the pressure chamber DK passes or merges from a limitation by the wall 3 into a limitation by the membrane 5. This can be well seen in FIG. 1 in that the pressure chamber DK below the arrow tip of the reference numeral line of the reference numeral 85 is only limited by the wall 3, above only by the membrane 5, which in turn cannot support itself against a section of the wall 3.

The fixing of the membrane 5 in the transition section 85 may be done in many ways. However, in this and in any other embodiments, it is not fixed by the connector 53. The fixing of the membrane 5 by the connector 53 takes place in the area of the connection A.

The floating 81 may be adjusted by either releasing or introducing fluid through the outlet or inlet 19′ in interaction with the connector 53 such that the contact point surface 83 and the transition section 85 are at the same height H (H is the height above the base area 4a) outlined by the dashed line in FIG. 1. Based on the experience of the inventor of the present invention, once this happens, optimal pressure conditions for taking the impression are thus present or provided.

Even if the connector 53 is not adjustable in its length, it advantageously ensures that a predetermined extent of floating 81 beyond the height H cannot occur. A predetermined extent may be 1 to 4 cm, particularly preferred about 2 cm.

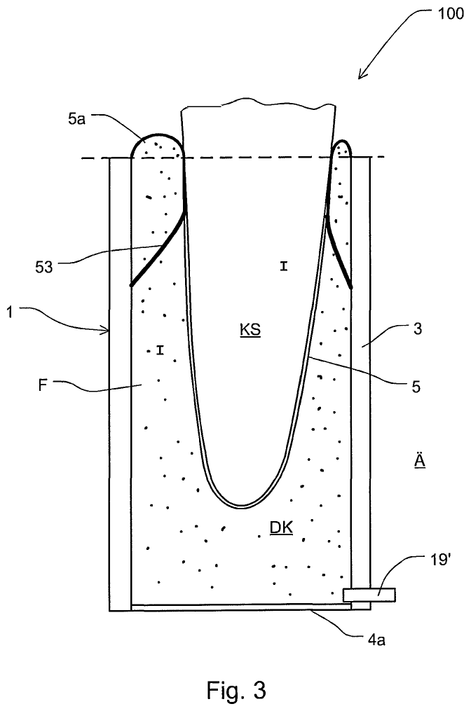

FIG. 1 shows a distal connector 91 which may be optionally provided-in any embodiment-although it is not shown in FIG. 3.

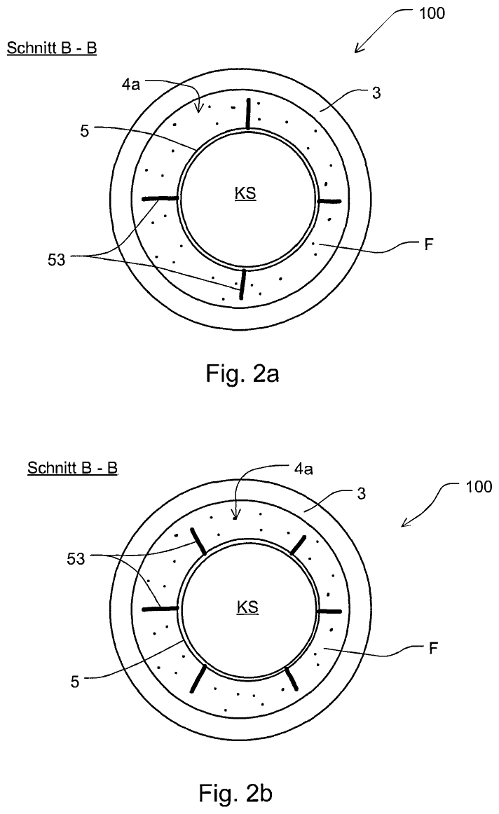

FIG. 2a shows the apparatus 100 according to the present invention of FIG. 1 in a further sectional plane B-B. The sectional plane B-B shows the apparatus 100 in cross section at a position in the longitudinal direction in which the connectors 53 are connected to the membrane 5. The view direction of the sectional plane B-B is directed in FIG. 2a downwards to the base area 4a. In FIG. 2a four connectors 53 are arranged each being offset by approximately 90 degrees in the circumferential direction. Alternatively, the connectors could be arranged irregularly about the circumference. They may also be placed in any number and at any distance from each other (about every 5 cm along the circumference).

The connectors 53 are fastened at one of their ends to the membrane 5, and at their other end preferably to the base area 4a. The limb stump KS is arranged in the center of the membrane 5.

The connectors 53 may be connected to each other, for instance in that they, for example all of them, radiate in an annular device or are connected to an annular device which is guided respectively around the circumference of the membrane 5.

FIG. 2b shows the arrangement of FIG. 2a, however with six connectors 53, which are each offset by approximately 60 degrees in the circumferential direction. Alternatively, the connectors could be arranged irregularly or scattered around the circumference.

FIG. 3 shows the longitudinally cut apparatus 100 according to the present invention in a further embodiment from the side. In this embodiment, the connectors 53 are fastened to the wall 3 of the pressure vessel 1. Functionally, the connectors 53 fastened to the wall have the same function, namely to prevent unwanted floating of the membrane 5. A fastening to the wall 3 may have advantages in terms of assembly and/or handling during use of the apparatus 100 according to the present invention.

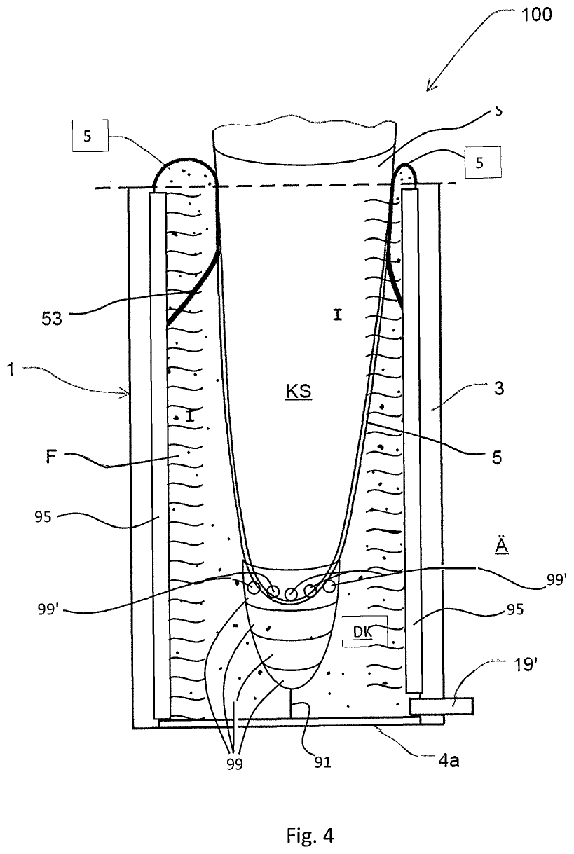

FIG. 4 shows the longitudinally cut medical apparatus 100 of the set according to the present invention in a further embodiment from the side.

Reference is made to the statements with regard to the preceding figures.

The membrane 5, as shown in FIG. 4, is filled due to the load by an inserted limb stump KS of a patient elastically into the depth of the interior I towards the base area 4a and, thus, abuts or contacts the latter, in particular without building up wrinkles or bulges.

On the left and right in the example of FIG. 4, light sources or UV light sources 95 are arranged as examples of energy sources or radiation sources within the pressure chamber DK, here exemplarily on the inner wall of the pressure chamber DK. Alternatively or additionally, they are arranged outside the pressure chamber DK and/or inside the pressure vessel 1, but not in the pressure chamber DK and/or not in the liquid F. Alternatively, they are provided outside the pressure chamber DK and/or outside the pressure vessel 1 and are not connected to the medical apparatus 100.

The UV light sources 95, should there be several thereof, as examples of energy sources or radiation sources may be arranged equally distributed around the circumference or at different distances from each other. For example, they are arranged over the entire length of the pressure chamber DK, i.e. in relation to the up-down direction of FIG. 4, or only parts thereof, and/or over the entire circumference, or only sections thereof.

In alternative or additional embodiments, the wall 3 of the pressure vessel 1 and its pressure chamber DK may have energy-permeable and/or translucent sections similar to windows, and the UV light sources 95 may be arranged outside the pressure vessel 1. This is also encompassed by the present invention.

The limb stump KS is mantled or covered with a stocking S, which in turn has been soaked in resin for creating a prosthesis shaft, or consists of resin and/or another curable material, or comprises resin and/or another curable material, before it is being inserted together with the limb stump KS through the opening of the pressure chamber DK into the latter and into the membrane 5.

An optional cup-shaped object, for example the vector tower 99, for example made of plastic, may be provided to be inserted together with the limb stump KS within the membrane 5, or to be present within the pressure chamber DK and thus in the fluid, and in either case to relieve the distal end of the limb stump KS from fluid pressure acting distally on it.

This object, if present as part of the set or apparatus, may comprise, e.g. at least in its uppermost segment or edge, holes 99′ or other radiation-permeable sections, in particular translucent sections, such as e.g. windows, here to allow light, in particular the UV light from the UV light sources 95, to penetrate to the tip of the limb stump KS.

The light radiation 97 from several UV light sources 95 is shown by wavy lines. It causes the resin or other suitable material with which the stocking S is soaked or which is otherwise placed around the limb stump KS to cure completely or at least partially. The so far cured stocking S may then be used directly as a prosthesis shaft or may be further processed as a blank for a prosthesis shaft, for example by thermal shaping and/or by attaching screw connections to further prosthesis sections or the like.

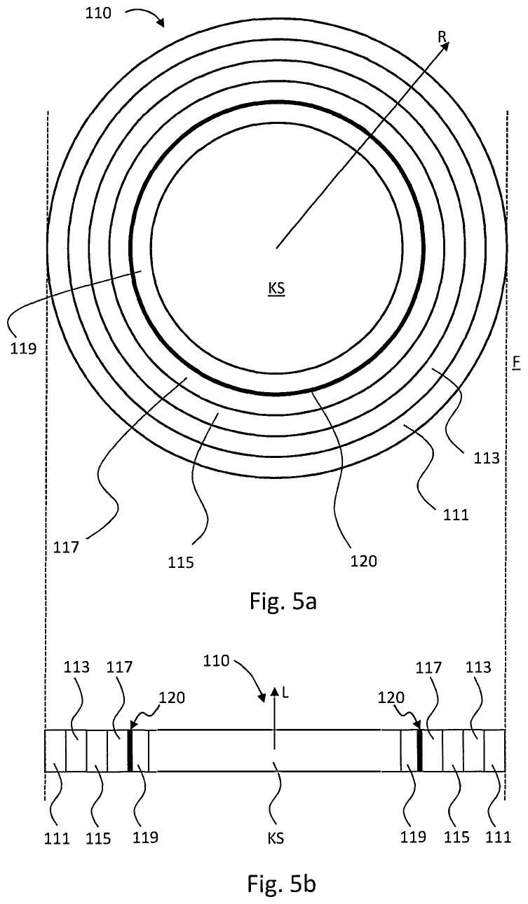

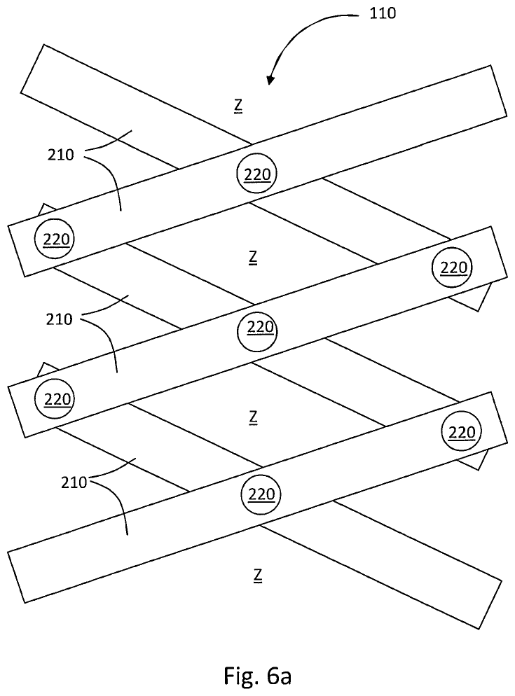

FIG. 5a shows, in a schematic representation, a braiding 110 according to the present invention (optionally the “sandwich structure” of a tube, a sock or a stocking, hereinafter referred to as braiding 110), in or on which a material or material according to the present invention, optionally the material of a set according to the present invention, is inserted or applied, in one embodiment.

A cross-section through the braiding 110 is shown, in its interior the limb stump KS lies within the pressure chamber DK during the fitting of the prosthesis, on the outside the fluid F acts on the braiding 110, separated by at least the membrane 5.

In the example of FIG. 5a, the braiding 110 is multi-layered, i.e. comprises different braided layers or fabric layers 111, 113, 115, 117, which are shown as circular layers (or plies) with different diameters for the sake of clarity. This representation is purely exemplary and should not be understood as limiting. Other shapes/outlines of the braiding are also encompassed by the present invention.

In some embodiments, two or more braided layers 111, 113, 115, 117 may comprise the same shape/outline, the same size and/or the same orientation.

In the example of FIG. 5a, the braiding comprises four braided layers 111, 113, 115, 117 as well as a textile layer, which may be e.g. a liner 119. The number and arrangement of the layers or braided layers are purely exemplary and are not to be understood as limiting.

In several embodiments, one or several of the layers, e.g. the braided layers 111, 113, 115, 117, comprise or consist of glass fibers.

Providing several layers, which lie on top of each other in the radial direction R, contributes to the fact that crossing points within the individual braided layers 111, 113, 115, 117, e.g. if these have the form of mesh, tend not to lie on top of each other in the entire braiding 110 or, if they do, this contributes to the fact that the resulting accumulation of material in the radial direction does not give rise to avoidable pressure points which impair the future wearing comfort for the patient or the process of generating.

In some embodiments, several of the layers comprise or consist of a mesh section or braided section.

Preferably, the optional liner 119 forms the innermost layer and is preferably made of or comprises an elastic fabric. Preferably, the optional liner 119 is a white fabric in order to increase the light yield. Preferably, in these embodiments, this liner 119 or this layer, which ultimately faces the patient's limb stump KS, is made of a very fine-meshed or fine-pored material in order to advantageously achieve the smoothest possible inner surface of the prosthesis shaft manufactured according to the present invention. This may contribute to achieving the later, preferably step-free inner side of the prosthesis shaft, which is required for the application of a vacuum and thus to achieving a good fit of the prosthesis shaft on the limb stump KS.

In certain embodiments, the liner 119 may be wet, for example soaked with water.

In the example of FIG. 5a, the optional liner 119 optionally forms the innermost layer of the braiding 110. Its outer side is exemplarily designed to be light-reflecting or reflective, for example by a mirror film 120 (see FIG. 5b for this).

FIG. 5b shows in a schematic representation the braiding 110 of FIG. 5a from the side, again in section, wherein only one longitudinal segment, not the entire braiding 110, is represented in its entire length. The entire braiding 110 would extend upwards and downwards beyond that shown in FIG. 5b, i.e. in the longitudinal direction L.

Reference is made to the description with regard to FIG. 5a in order to avoid repetition.

An optional mirror film 120, not shown in FIG. 5a, can be seen-preferably between the outermost of the braided layers 111, 113, 115, 117, and the liner 119. The mirror film 120 may in particular increase the luminosity e.g. of the UV lamps and improve the penetration of the, optionally several, deformable braided layers 111, 113, 115, 117 which are curable by an energy source. This may also contribute to shortening the curing time of the material according to the present invention.

In some embodiments, the material according to the present invention, which may be incorporated into or applied onto the braiding 110 as shown in FIG. 5a and FIG. 5b, is, or comprises, a light-curing resin, preferably an acrylic resin. It may be, or comprise, a photopolymer. It may comprise reactive additives and/or an odorant.

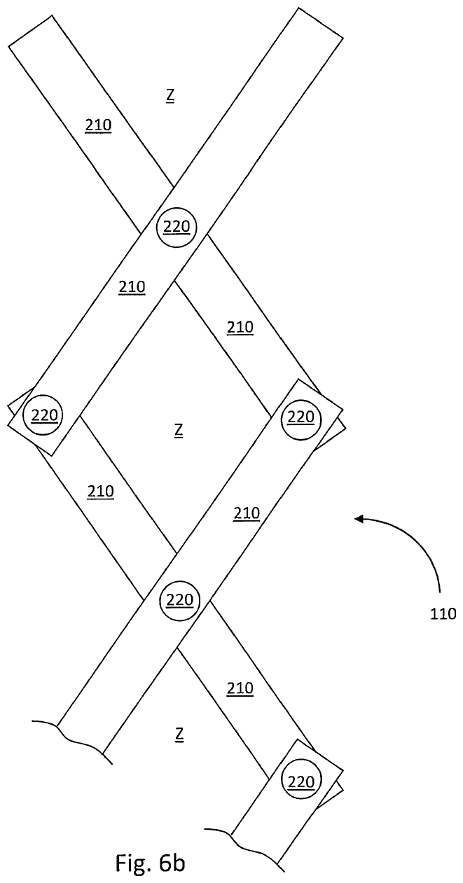

FIGS. 6a and 6b show, in schematical representation, the possible principle of the braiding 110, or only individual layers thereof, again in another embodiment.

In this, the braiding 110 is shown by elongated or long-stretched mesh structure sections or braided structure sections 210 (hereinafter referred to as sections 210 for short), which are intended to embody or represent fibers or threads or strips. The elongate sections 210 are connected at one or more, e.g., end sections and/or central sections, referred to here as cross points or knots 220, to at least one other elongated section 210, for example to at least one adjacent elongated section 210, in particular in a form-fit and/or force-fit manner.

However, between such crossing points or knots 220, the sections 210 connected in them are preferably spaced apart from adjacent sections 210, outlined by the space Z in

FIGS. 6a and 6b, which ultimately results in the mesh structure with individual spaces Z respectively enclosed.

In the example of FIGS. 6a and 6b, the crossing points 220 are preferably knots between fibers and/or threads, represented by the sections 210, wherein a section 210 may be connected to one, two or more other sections 210.

The comparison of FIG. 6a with FIG. 6b shows the optional scissor-like or diamond-like deformability of the arrangement of the sections 210 by the crossing points 220, wherein a relative movement between two sections in contact with each other at a specific crossing point 220 is not required and, therefore, may or may not be permitted.

Providing several layers or braided layers 111, 113, 115, 117 of such braiding 110, which come to lie on top of one another in the radial direction, contributes to the fact that the crossing points 220, e.g. of the mesh, of these layers are unlikely to lie on top of one another, or only in a few cases, or if they do, the resulting accumulation of material in the radial direction gives no rise or reason for avoidable pressure points which impair, for the patient, the wearing comfort of the prosthesis shaft generated according to the present invention or the process of generating said prosthesis shaft.

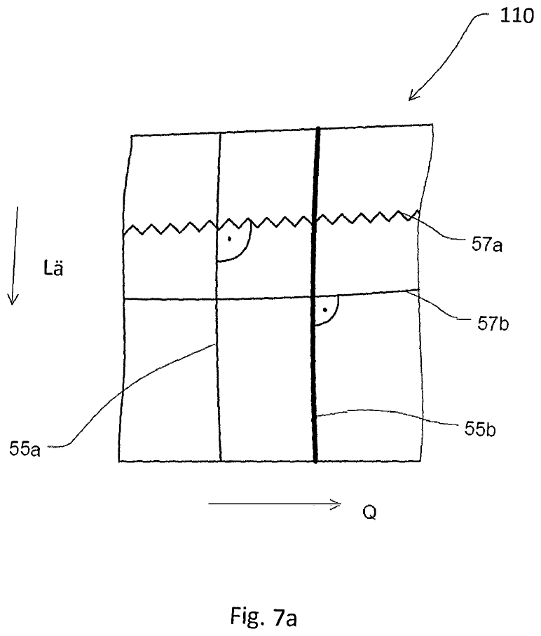

FIG. 7a shows schematically very simplified and again only a section of a braiding 110 in a further embodiment.

The braiding 110 is shown in the cut-open state, in which it does not assume the shape in which it is used in the apparatus 100 or in the set according to the present invention. It could be brought into the shape, exemplarily shown or at least outlined in FIG. 5a, by joining its left and right edges together and tailoring or cropping it where necessary.

In FIG. 7a, it is assumed that the braiding 110 comprises only one braided layer as disclosed herein and e.g. denoted with 111, 113, 115 or 117 in FIG. 5a. However, what is shown in FIG. 7a may also apply to or be generalized to braidings 110 with more than one braided layer. For reasons of clarity solely, exactly one braided layer is shown in FIG. 7a.

Two pairs of longitudinal fibers and transverse fibers or longitudinal strips or transverse strips can be seen purely exemplarily, which are respectively denoted with 55a and 57a and as 55b and 57b, which are here only exemplarily perpendicular to each other. What is stated above with regard to the sections 210 and does not contradict what is disclosed here with regard to the fibers or strips 55a or 57a and as 55b and 57b may also apply to the latter, i.e. the fibers or strips 55a or 57a and 55b or 57b.

The longitudinal fiber 55a is optionally stretched and, therefore, optionally does not allow any further stretching in the longitudinal direction Lä.

In contrast to this, the transverse fiber 57a to be considered in connection with it may allow stretching of the braiding 110 in the transverse direction Q due to its looped or twisted or corrugated course or material in the matrix of the braiding 110, which, in addition to the fibers, may for example also be soaked with the material according to the present invention or may have absorbed the material. Thus, the longitudinal fiber 55a and the transverse fiber 57a differ from each other in their ability to stretch or unfold or extend under tensile load, e.g. functionally; optionally they differ also with regard to the material from which they are made and/or with regard to their cross-section.

In the two exemplarily considered fibers 55b and 57b, which belong to each other, the same or a similar effect may be achieved differently. The longitudinal fiber 55b is thick and may therefore not stretch or, compared to the associated thinner transverse fiber 57b, stretch less.

It goes without saying that the braiding 110 may comprise more fibers than those shown here. Also needless to mention that the braiding 110 may preferably comprise longitudinal fibers and transverse fibers 55a and 57a or longitudinal fibers and transverse fibers 55b and 57b, preferably therefore not necessarily both pairs of the longitudinal or transverse fibers discussed herein.

FIG. 7b and FIG. 7c show a braiding 110 with one or more braided layers having longitudinal fibers 55 and transverse fibers 57, similar to the representation of FIG. 7a, wherein the transverse fibers 57 connect the longitudinal fibers 55.

The longitudinal fibers 55 run straight, while the transverse fibers 57 run between adjacent (alternatively between non-adjacent) longitudinal fibers 55 with both transverse and longitudinal extension.

The transverse fibers 57 thus run for example, as shown in FIG. 7b and FIG. 7c, in a zigzag pattern. They may assume a single zigzag course, as in FIG. 7b, a double zigzag course, as in FIG. 7c, or a multiple zigzag course, as not shown, or loop or meander otherwise.

In this, the transverse fibers 57 and the longitudinal fibers 55 may certainly have the same structure and/or the same thickness. It is their arrangement with regard to the longitudinal direction that makes the braiding 110 (in the transverse direction Q) stretchable or extendable (or more stretchable or more extendable) or (in the longitudinal direction Lä) not stretchable or extendable (or less stretchable or less extendable).

If the longitudinal fibers 55—unlike shown in FIGS. 7b and 7c—are not allowed to run in a straight line, but are instead allowed to run in a more or less pronounced zigzag around a straight line in the longitudinal direction Lä, a shortening of the braiding 110 in the longitudinal direction may be advantageously achieved if desired. Thereby, a possible shortening may be “set” or limited in advance by coordination of dimensions and measurements of the zigzag pattern of the longitudinal fibers 55 and/or the transverse fibers 57. A shortening may be advantageous insofar an undesired elongation of the braiding 110 may be simply and yet effectively counteracted structurally.

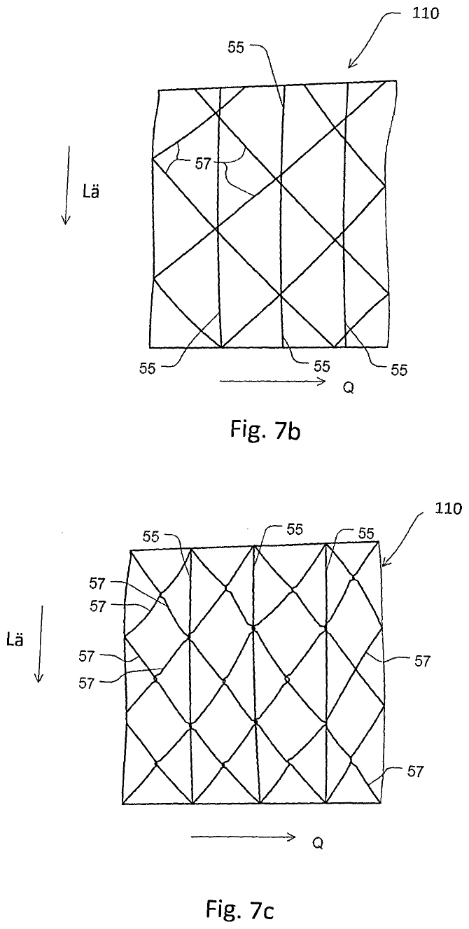

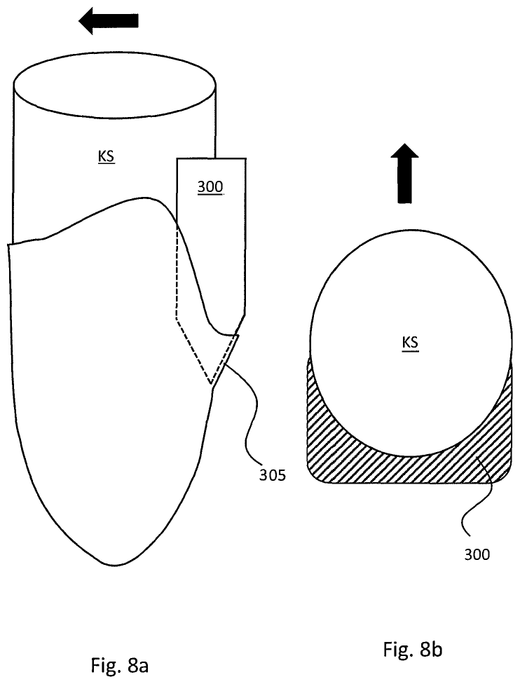

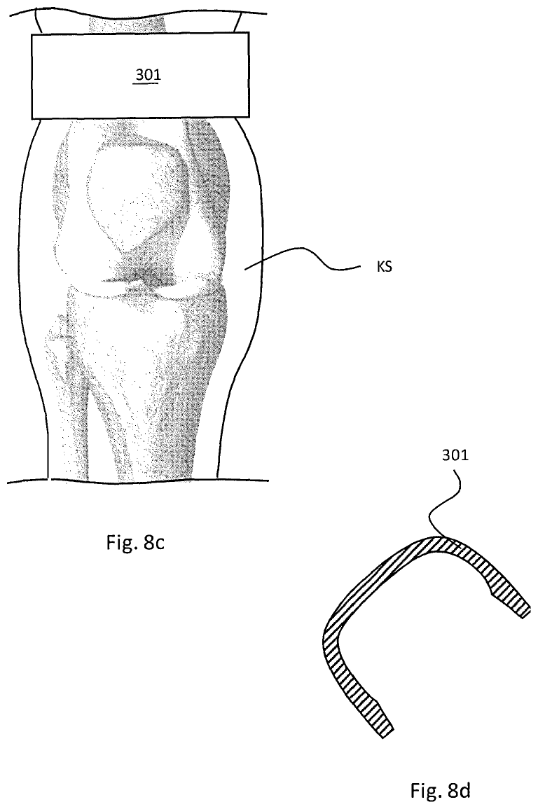

FIG. 8a shows, schematically simplified, a form body 300 in a first embodiment, which may be a component of the set or kit according to the present invention and may be used in the method according to the present invention. Such a form body 300 may, for example, be made of silicone or another suitable material.

In the example of FIG. 8a, the form body 300 is applied from posterior to the patient's limb stump KS and optionally fixed to it, for example by film.

Its design may be used to widen the prosthesis shaft to be created posteriorly or dorsally, e.g. similar to the upper edge of a calyx, in order to allow subsequent relief of the flexor tendons of the patient wearing the prosthesis shaft. The widened area is denoted with 305 in FIG. 8a.

As in FIG. 8b, the arrow here points from posterior or dorsal to anterior or ventral.

FIG. 8b shows, schematically simplified, a cross-section of the limb stump KS with the attached or laid form body 300 of FIG. 8a from above.

FIG. 8c shows, schematically simplified, the knee joint of a patient with a form body 301 herein attached proximally in a further embodiment, which may be a component of the set or kit according to the present invention and may be used in the method according to the present invention. Such a form body 301 may, for example, be made of or comprise plastic, reinforced or carbon-reinforced plastic or another suitable material, in particular as disclosed herein.

In the example of FIG. 8c, the form body 300 optionally has the shape of a brace, which is attached from anterior proximal to the patient's knee joint. The form body 301 serves to narrow the limb stump KS above the knee joint or to compress soft parts during the creating of the prosthesis shaft or the plaster cast in order to increase the wearing comfort for the patient of the prosthesis manufactured on the basis of the limb stump KS compressed in this way, wherein it is the same whether the prosthesis is manufactured from curable material or conventionally, that is e.g. following a plaster cast. Such compression may be, e.g., between 6 mm and 7 mm per side (lateral, medial).

FIG. 8d shows, schematically simplified, a top view of the form body 301 of FIG. 8c.

A form body 300, 301 may be placed from anterior or ventral or from posterior or dorsal on the limb stump KS. The embodiments of FIGS. 8a to 8d are purely exemplary and are not to be understood as limiting.

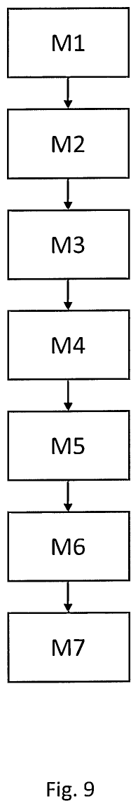

FIG. 9 shows, schematically simplified, an exemplary course of a method according to the present invention for adapting a deformable material which is curable by an energy source to a limb stump KS or for creating a prosthesis shaft or a part thereof for a limb stump KS of a patient in a first embodiment.

Reference is made to the reference numerals with regard to the aforementioned figures.

The method step M1 represents providing a set according to the present invention.

Filling the fluid chamber or pressure chamber DK of the pressure vessel 1 with a fluid as disclosed herein is represented by method step M2. Alternatively, this method step M2 represents providing a fluid chamber or pressure chamber DK filled in this way.

In method step M3, there is shown an insertion of the limb stump KS into the membrane 5. This insertion takes place such that the limb stump KS is, at least in sections thereof, surrounded by the membrane 5 around its entire circumference, and such that the deformable material which is curable by an energy source is present at least in sections between the limb stump KS and the membrane 5. Thereby, the limb stump KS may alternatively be surrounded or wrapped, at least in sections, with a moist plaster bandage. The method step M3 may optionally also encompass placing a form body 300, 301 on the patient's skin.

Optionally, a curing or partial curing of the material by using the energy source inside the pressure vessel 1 is represented in method step M4.

An optional subsequent removal of the limb stump KS, preferably together with the optionally fully or partially cured material surrounding it, from or out of the pressure vessel 1 is represented in method step M5.

An optional or further curing, or partial curing, of the material using the energy source outside the pressure vessel 1 is represented in method step M6. This step may be carried out in addition to or as an alternative to method step M4.

An optional creation of a complete prosthesis based on the thus achieved prosthesis shaft, or a respective part thereof, based on the cured material, without having previously taken a positive impression of the limb stump KS is represented in the method step M7, or based thereon.

Although the present description focuses on creating a prosthesis shaft based on a curable material, the present invention is not limited thereto. A number of combinations of features and embodiments are also according to the present invention, although they are based on conventional (negative) plaster impressions. For example, the use of form bodies as described herein may thus be claimed as an invention. Their use is not limited to the simultaneous use of curable material.

### LIST OF REFERENCE NUMERALS

100 apparatus
1 pressure vessel
2 first end side
3 wall
4 second end side, may optionally be closed or sealed with the base area
4a base area
5 membrane
7 upper edge
5a proximal or first membrane section; reinforced section of the membrane
5b distal or second membrane section
9 insertion opening
19′ outlet, inlet
53 connector
55 longitudinal fiber
55a longitudinal fiber
55b longitudinal fiber
57 cross fiber
57a cross fiber
57b cross fiber
58 central or middle area or section of the membrane
81 floating of the membrane
83 contact point surface
85 transition section
91 distal connector
95 UV light source
97 UV light radiation
99 vector tower
991 holes; translucent sections
110 braiding; tube; sock; stocking
111 braided layer; fabric layer
113 braided layer; fabric layer
115 braided layer; fabric layer
117 braided layer; fabric layer
119 liner
120 mirror film
210 mesh structure section; braided structure section; section
220 crossing points; knots
300 form body
301 form body
305 expanded site of the prosthesis shaft
A connector
Ä exterior of the pressure vessel
B distance
DK pressure chamber of the pressure vessel
I interior of the pressure vessel
F fluid or liquid
H height of the pressure chamber
KS limb stump
L longitudinal direction
Lä longitudinal direction of the braiding
M1 bis M7 method steps
Z transverse direction of the braiding
R radial direction

1. A set for use in taking an impression of a limb stump, or in generating a prosthesis shaft or a part thereof, wherein the set comprises or consists of:
a medical apparatus, wherein the apparatus comprises at least:
a pressure vessel with a fluid chamber or a pressure chamber for receiving or storing a fluid, in particular one being under pressure, wherein the pressure vessel comprises a wall made of a first material, wherein the wall limits an interior of the pressure vessel against an exterior of the pressure vessel, wherein the pressure vessel comprises an insertion opening for inserting the limb stump into the interior of the pressure vessel;
a fluid-tight membrane made of at least a second material, which membrane is arranged to form or limit the fluid chamber or the pressure chamber;
and
at least one energy source for curing or partially curing a deformable material in order to obtain or achieve an impression of the limb stump or to obtain or achieve a prosthesis stump; and/or
a deformable material which may be cured by an energy source and which is or comprises a light-curing resin or a photopolymer.

2. The set according to claim 1, and wherein the at least one energy source is, or comprises, a radiation source.

3. (canceled)

4. The set according to claim 2, wherein theradiation source emits radiation with a wavelength between 395 nm and 415 nm.

5. The set according to claim 1, wherein the at least one energy source for the curing or the partial curing is connected to a section of the pressure vessel.

6. The set according to claim 1, wherein the at least one energy source for the curing or the partial curing is detached from the medical apparatus or at least from a section of the pressure vessel.

7. The set according to claim 1, wherein the at least one energy source is arranged outside the pressure chamber, the pressure vessel or the medical apparatus.

8. (canceled)

9. The set according to claim 1, wherein the medical apparatus or sections thereof, comprises at least one section which is permeable to radiation comprising light radiation or UV light radiation.

10. The set according to claim 9, wherein the sections, of the medical apparatus comprise, or consist of, an UV-resistant acrylic glass.

11-14. (canceled)

15. The set according to claim 1, wherein the deformable material is applied to and/or respectively incorporated into a braiding, a mesh structure, a tube, a sock or a stocking.

16. The set according to claim 15, wherein the braiding, the tube, the sock and/or the stocking comprises several layers.

17-20. (canceled)

21. The set according to claim 1, comprising a liner which is light-reflecting or reflective on an outer side of the liner.

22. The set according to claim 16, wherein an innermost layer of the layers is light-reflecting or reflective on an outer side of the innermost layer.

23. (canceled)

24. The set according to claim 1, comprising a liner having light sources or being prepared to direct or guide light into the liner.

25-28. (canceled)

29. The set according to claim 1, wherein the membrane of the medical apparatus is, at least in sections, permeable to rays of the energy source.

30. The set according to claim 15, with a control device configured to limit the energy source or its power or output such that a temperature of 40° C. is not exceeded on the inside of the braiding, the tube, the sock or the stocking.

31. A method for fitting a material being deformable and curable by an energy source to a limb stump, or for creating a prosthesis shaft, or part thereof, for a limb stump of a patient, the method encompassing the steps of:
providing a set according to claim 1;
filling the fluid chamber or the pressure chamber of the pressure vessel-with the fluid, or changing a liquid level within the fluid chamber or the pressure chamber, such that the membrane, at least in sections thereof, is covered with or by the fluid around the entire circumference of these sections, or such that the membrane cambers through the insertion opening of the pressure vessel outwardly into the exterior of the pressure vessel, or providing the fluid chamber or the pressure chamber filled in this way.

32. The method according to claim 31, having the further step:
inserting the limb stump, into the membrane such that the limb stump is, at least in sections thereof, covered or surrounded or enclosed by the membrane around its entire circumference, and such that the deformable material which may be cured by the energy source is present at least in sections between the limb stump and the membrane.

33. (canceled)

34. The method according to claim 31, having the further steps:
removing the limb stump, preferably together with the deformable material surrounding it, from the pressure vessel; and
curing, or partially curing, the deformable material using the energy source outside the pressure vessel.

35. The method according to claim 31, having the further step:
curing, or partially curing, the deformable material using the energy source within the pressure vessel; and
removing the limb stump from the pressure vessel.

36. The method according to claim 31, having the further step:
introducing energy into the liner or along the liner material.

37-39. (canceled)

40. A material which is deformable and curable by an energy source,
wherein the material is, or comprises, light-curable resin, preferably acrylic resin; and/or
wherein the material is, or comprises, a photopolymer; and/or
wherein the material comprises an odorant; and/or
wherein the material comprises reactive additives; and/or
wherein the material is applied to and/or incorporated into a braiding, a tube, a sock or a stocking; optionally, wherein the braiding, the tube, the sock and/or the stocking are multilayered; optionally, wherein the braiding, the tube, the sock and/or the stocking comprises several layers, preferably at least 4, preferably at least 5 or 6 layers; optionally, wherein several of the layers comprise, or consist of, glass fibers; optionally, wherein the glass fibers are, or can be, connected to an energy source, for example to a light source, by connectors, connections or the like, in order to guide light, in particular UV light; optionally, wherein an outer sheath of the glass fibers comprises, at least in sections apertures, windows and or the like to allow light, in particular UV light, to be emitted from the fibers for prompting the curing of the material; optionally, wherein several of the layers comprise, or consist of, a mesh section or braided section; and optionally, wherein one of the layers, in particular the innermost layer, is made of, or comprises, an elastic fabric.

41-57. (canceled)

58. The set according to claim 9, wherein the sections of the medical apparatus which are permeable to the radiation comprise the wall of the pressure vessel or an area of the wall of the pressure vessel.
